(19) United States
(12) Patent Application Publication
(10) Pub. No.: US 2005/0096733 A1
(43) Pub. Date: May 5, 2005

(54) Stent and method for the production thereof (variants)

(76) Inventors: July K Kovneristy, Moscow (RU); Boris A. Goncharenko, Moscow (RU); Oleg A. Vytouley, Moscow (RU); Anatoly K. Kulapov, Moscow (RU); Valery A. Cherkasov, Moscow (RU); Alexei V. Ivanov, Moscow (RU); Victor T. Partosh, Moscow (RU)

Correspondence Address: STAAS & HALSEY LLP, SUITE 700, 1201 NEW YORK AVENUE, N.W., WASHINGTON, DC, 20005, US

(21) Appl. No.: 10/500,294

(22) PCT Filed: Dec. 29, 2001
(86) PCT No.: PCT/RU01/00577

(51) Int. Cl. A61F 2/06

(52) U.S. Cl. 623/1.22

(57) ABSTRACT

The inventions relate to the field of medicine and can be used in endoprosthetics for restoring a lumen in narrowed sections of vessels and other hollow organs. The stent is made as a netted hollow volumetric body, formed by interweaving of several groups of windings from a single length of a thread, placed along helical spirals with opposite entry directions. A material of the thread possesses elasticity and shape memory effect. Cells which are opposite with respect to the axis of the stent, are displaced with respect to each other, and the stoat thread has different elasticity in its separate sections, preserving the same thickness of the thread along the whole length of the stent. In the second variant of the stent its geometry is changed by way of making the stent with separate areas having a different axial curvature. The third variant of the stent unites the first and the second variants of the stent. In addition to that in all three variants the stent is manufactured from a medical nitinol Ti Ni. The method of stent manufacturing includes the formation of a netted hollow metallic body from a metallic thread by interweaving its windings, wound on a mandrel made as a body of revolution with a rectlinear longitudinal axis, and a pre-deformation of the stent on the mandrel by way of its quenching from the temperature 630-660° C. into water, which attributes a maximum elasticity to this stent. After the mandrel is removed the elasticity of separate sections of the stent is reduced, subjecting them to a secondary heat treatment at the temperature of 330-550° C. during from 1 to 30 minutes. In the rest variants of the method, the pre-deformation of the stent is conducted twice, first by way of primary heat treatment of the stent on the mandrel with a rectilinear longitudinal axis at the temperature of 330-390° C. during 5-20 minutes, and then on the mandrel with a curvilinear longitudinal axis, the form of which corresponds to the form of a prostheticated vessel. By that, by changing the time and temperature of the secondary heat treatment, the elasticity of the thread in separate sections of the stent and/or axial curvature of the stent are changed.

### FIELD OF THE ART

The proposed invention relates to the field of medicine and can be used while making endoprostheses for restoring a lumen in narrowed sections of vessels and other hollow organs.

### PRIOR ART

There is a great number of pathologies causing a stenosis or full on ion of vessels or other hollow org, as a result of which their functioning is partially or completely disturbed, which in its turn can be the reason of such serious diseases as insultus, infarctions, etc., up to a lethal outcome.

A wide spreading of such types of diseases requires the development of effective methods for their treatment. One of such most widely spread method of treatment is the use of hollow endoprostheses, called stents. As a rule, they represent a hollow cylindrical body of revolution, which is introduced into a vessel or other hollow organ is fixed in a required place, and maintains a necessary lumen in an organ.

The stents should satisfy to numerous requirement, some of which are in a certain contradiction to each other, which hinders a search for optimal solutions.

The stents should provide;
effective restoration and support of lumens of vessels and other hollow organs;
biological compatibility with body tissues;
a possibility of using in different anatomic zones of hollow organs;
a possibility of using in a broad range of intervals;
a minimum traumatism during conducting an operation and bringing post-operational complications down to a minimum;
a possibility of using a simple and safe stent delivery system;
a possibility of controlling an operation for installation of a stent with a help of an x-ray and other radiation.

In addition to that, there exists many other additional requirements, among which it is necessary to point out an undesirability of using expensive technologies and materials for stent manufacturing, which increase the cost of an operation and is an obstacle for a mass application of endoprosthetics.

All these conditions impose rather strict conditions upon selection of stents design, which could be recommended for the use in medical practice.

At present there exists a great number of variable sty designs, the most wide spread of which are stents made as hollow bodies of revolution, the surface of which has a lattice or cell structure. Both non-metallic and metallic materials are used for the manufacturing of a stent. During selection of the material, in addition to other conditions, the preference is given to the materials which are opaque for an x-ray and other radiation, used in endoprosthetics for the control for the course of an operation.

Among metallic materials one should point out a wide use of different metals and alloys having a high elasticity and/or shape memory effect, such as stainless steels, tantalum based alloys, titanium-nickel alloys, etc. Out of titanium-nickel alloys one should specifically single out the use of the soled “medical nitinol”—TiNi allow, containing 55.5-56.0 Ni in weight percent, the rest being Ti. The selection of an alloy and the relationship of its components are to a great extent determined by a temperature range, characteristic of this alloy, at which the stent manufactured from it recovers an initially specified form. The practice of implanting nitinol stents into a vascular system, gastroenteric tract, uropoietic organs, hepato-biliary system, etc., is known at present.

During the manufacturing of stets with a lattice or cell surface, cylindrical mandrels became widely used, onto which metallic threads are wound, both the ones attributing a certain shape to them and not doing that, which are connected to each other either by way of braiding or by some other method, forming stent cells.

In the majority of known stents the task of providing a uniform pressure upon a surface of an organ, for which a prosthesis is made, is being solves. However, the necessity of solving an opposite tasks, that is of providing an increased or decreased pressure in separate sections of the organ being prostheticated has to be solved. Such task can arise, for example, when there is an adenoma or when a stent is installed in a bent section of an organ for which a prosthesis is made. Even more so, there can be several sections of this type and they might have a different curvature. In this case a stent should assume a bent form. However, if the stent has the same elasticity along its all length and its geometrical compatibility with bent sections of organs being prostheticated is only due to the stent elasticity, then the bent sections of the organ being prostheticated shall be subjected to higher loads than its rectilinear sections, as additional elastic forces, arising when bending moments affect the stent, shall act upon them. Naturally, the above negative effects can be avoided to a considerable extent, if one uses the way of implanting several short stents, which will not cause an expressed deformation of vessels. But it complicates the course of operation and considerably increases expenditures for it; that is why this way has not found a wide application. In many cases an additional difficulty is also caused by the necessity of using a small diameter catheter during a delivery and installation of the stent. Such situation can arise when the organ itself for which a prosthesis is made does not have bents and small diameter, but one has to use narrow and bent vessels for the delivery of the stent to the place of its installation. An additional problem arises while designing small diameter stents due to the necessity of using a small diameter wire for their manufacturing, which requires providing for a necessary rigidity.

Thus, in order to overcome the above difficulties, it is necessary to develop such stents which, on the one hand, should create a same for deformed vessels, bile ducts, ducts and other tubular organs, and, on the other hand, would repeat the form of their bents without a rough straightening of organs being prostheticated, which can cause a distortion of their normal functioning. Main arteries and pelvis veins, arteries of a kidney, esophago-gastral pass, intestine, different sorts of stenosis of vascular, gastroenteric, biliary-digestive, and other types of anastomosis, etc., are the most vulnerable in this respect. Besides, the design of the stents should provide for their convenient delivery to an organ being prostheticated.

These tasks are solved by different design solutions in the known stents.

So, known is a stent, representing a braided design, formed of a plurality of rows of rhomboidal rigidly connected cells having a different axes dimensions ratio. Each of the cells is connected by apexes by means of inseparable units (patent RU 2157146, A 61F2/04, 1995). By that, the cell is a rigidly closed functional element and can have both a rectangular and trapezoidal form. A rigid connection of the cells can be implemented by rigidly interwoven intergripping loops. A possibility of braiding the stent from a continuous thread (wire) is provided. The design of the stent allows to change its axial and radial rigidity in wide limits by changing a cross section of a thread and a number of rows. The stent can be made in the form of different bodies of revolution with different diameter along its led which allows to change its elastic properties along the stent length. However, geometrical dimensions of the cells will also change with that, which spoils a homogeneity of its reaction to the installation of the stent in a zone of a prostheticated organ contact with the stent, which can be the reason of post-operational complications. In addition to that, an availability of a big number of rigid connection nodes of cells which render an undesirable local action upon a vessel for which a prosthesis is made, can be the reason of complications. The above rigid nodes also decrease the stent transformation possibilities, which limits the sphere of its application. By that, this design does not have a high adaptability to a (streamlined) manufacturing.

The above drawbacks are partially eliminated in the design of the stents, a frame of which is formed by interweaving of at least two groups of coils of a tad (wire), placed along helical spirals with opposite entry directions.

Thus, known is a stent having a cylindrical form which formed is formed by an interweaving of a multitude of wire sections, helically wound with the opposite entry directions (patent U.S. Pat. No. 5,575,818. A 61 F 2/06, 1995). The stent elasticity can be different along the length of the stent due to changing a pitch of wire winding or due to changing a diameter of the stent at its separate sections. However, these changes are foreseen only at the end sections of the stent. The stent is manufactured from cobalt-chromium-nickel alloy or from stainless steel. This stent is distinguished by a better transformability compared to the preceding analogue; however it also does not allow to provide for a homogeneity of a reaction of an organ for which a prosthesis is made in the place of the stent installation. Besides, it also does not solve problems, related to the installation of the stent in bent sections of organs for which a prosthesis is being made.

Known is a stent made as a volumetric body with a surface formed by at least two groups of interwoven threads, placed as multiply entry turns along helical spirals with opposite entry directions latent RU 2053734, A61F2/06, 1992). The volumetric body has a variable transversal diameter and is made out of the material having a shape memory effect, such, as for example, nitinol. Elastic properties of the stent can vary along the stent length due to changes in its diameter. The stent has a good transformability, as the threads of different groups can slide with respect to each other, changing the diameter of the volumetric body. However, it also does not completely eliminate all the drawbacks inherent to the preceding analogue.

Known is a stent made in the form of a zigzag wire spring of a nitinol thread, having the form of a cylindrical spiral, each turn of which is formed of a specified and identical number of zigzags (patent RU 2063249, A61M29/00, 1990). By that, bents of one spiral turns aro located in the gaps of other spiral turns, opposite to each other. This design of the stent is meant for facilitating a geometrical compatibility of the stent with curvilinear sections of hollow organs. The elasticity of the stent varies by changing the “density” of the spiral pattern, however, a differentiated variation of elastic properties along the stent length is nowhere mentioned in the patent. Besides, in such design of the stent, the “density” of a spiral pattern will be different at convex and concave sections of the stent, which will cause a non-homogeneity of the reaction of an organ for which a prosthesis is made in the zone of the stent installation. It should be also pointed out that common drawbacks of spring stents, requiring, where it is needed, a uniform pressure upon a vessel wall, a small pitch of a spring, deteriorating an ion exchange, but not providing for enough uniformity of pressure, are inherent to the above design.

Known is a stent comprising at least two rigid segments, made in the form of a cylindrical metallic lattice with cells and a flexible connector, having numerous flexible joints, connecting apexes of adjacent cells at neighbouring rigid segments (patent RU2154443, A61F2/02, 1995, PCT/US 95/05095). It is recommended to manufacture the stent out of rather plastic, than elastic, biocompatible materials with a small shape memory effect. The design of the stent allows to use it at bent sections of prostheticated organs as well as to provide different elasticity at separate sections of the stent. However different dimensionss and the form of cells of rigid segments and of the connector do not permit to provide for a homogeneity of a reaction of an organ for which a prosthesis is made in the zone of the stent installation. The stent also have restrictions in part of its transformation, which interferes with the use of the stent in narrow vessels, and requires a rather complex manufacturing technology.

Known is a stent comprising a wire frame of a cylindrical form made of nitinol (patent RU2120253, A61F2/06, 1993; PCT/WO 94/03127). A surface of the frame in the development is formed of many rows of cells connected with each other. Each cell comprises two U-shaped wire elements, forming together with each other approximately an elongated oval, a big axis of which is directed along a cylinder circumference, and a smaller one is parallel to the stent axis. Adjacent cells in the neighbouring rows are displaced with respect to each other along the axes of the oval. A typical row of closed cells comprises at least one closed, circular form, U-shaped wire element. The adjacent cells in the above row are flexibly connected to each other, for example, by interweaving their branches. The design of the stent provides for the stent rigidity in a radial direction and allows it to be rather well adjusted to the geometry of hollow organs when the action of elastic forces, appearing during the action of bending moments upon the stent, decreases. By changing a total area of a stent elements cross-section, itis possible to change its axial rigidity, as well as to change a radial and axial rigidity of the stent, by changing a wire element cross-section. By that, it is, however, impossible to achieve the changing of the stent rigidity at its separate sections, as it will be the same along all length of the stent. Besides, the proposed stent design will experience difficulties in transformation because of the presence of relatively rigid element in the frame—a circular row of cells, which will hardly allow to use it in prostheticated organs with small diameters. It should be also pointed out at this design requires a rather big labour input for its manufacturing.

Known is a method of stent manufacturing, in which a sinusoidal wave structure is formed from a continuous wire; it is wound along a helical line onto a cylindrical mandrel and apexes of adjacent turns are connected, forming closed cells, of which a cylindrical surface of the stent is formed (patent U.S. Pat. No. 5,443,498, A61F2/06, 1993). The stent is manufactured from a material having a low level of shape memory effect, and after the frame of the stent is finished, it can be annealed with the aim of reducing tensions, arising in the wire in the process of the stent manufacturing. The proposed technology allows to manufacture stents characterised by small traumatism for prostheticated organs, relatively good transformability, and sufficient radial rigidity. At the same time this technology requires a relatively big labour input and is not meant for manufacturing the so-called “self-opening” stents. It also does not provide for the manufacturing of stents, which separate sections have different elasticity, as well as for manufacturing of stents having a curvilinear form in the operating condition.

Known is a method of stent manufacturing, in which sinusoidal wave structures are formed out of two sections of a wire, which are wound onto a cylindrical mandrel and apexes of adjacent turns of wave structures are connected by way of weaving, forming closed cells from which a cylindrical surface of the stent is formed (patent U.S. Pat. No. 5,913,896, A 61F2/06, 1997). The stent is manufactured from biocompatible materials, having a high corrosion resistance and good plasticity. According to the authors' opinion the following materials can be referred to such materials; tantalum, stainless steel, titanium, niobium, gold, nickel-titanium alloys, including nitinol, etc. The above method is characterised by a greater adaptability to manufacture than the method of patent U.S. Pat. No. 5,443,498, however it also does not provide for the manufacturing of stents, separate sections of which can have different elasticity, as well as for the manufacturing of stents with a curvilinear form. Besides, the presence of a great number of twists reduces the stent transformability and its biological compatibility with an organ being prostheticated.

The closest analogue to all variants of the claimed stent, selected as a prototype, is the stent made in the form of a netted hollow volumetric body, formed by interweaving of at least two groups of turns, placed along helical spirals with opposite entry directions, made of a single length of a thread (patent RU 2089131, A 61F2101, 2/06, 1993, patent U.S. Pat. No. 6,007,574, A61F2/06, 1994; patent EP 737452, A61F2/06, 1994). The material of the tad has elasticity and shape-memory effect. The design of the stent foresees a possibility of a differential changing of an elasticity of the stent at its separate sections at the expense of changing a spiral ms pitch, transversal diameter of the stent or additionally wound threads. It provides for a necessary rigidity of the stent, including that at its face parts, high elasticity and sufficiently high degree of transformability. This design has a good adaptability to manufacture. However, when changing the stent elasticity at its separate sections by proposed means, invariability and homogeneity of cell dimensions is not provided which can become the reason of spoiling a function of a prostheticated organ due to the danger of thrombus formation, appearance of stenosis and other post-operational complications. Besides, there exists a necessity in the increasing of a stent transformability, as well as giving a curvilinear form to it when required, which will provide for a possibility of using the stent, by way of its transportation or installation, in organs with a small cross-section and/or having a curvilinear form.

The closest analogue to both variants of the claimed method of manufacturing a stent, selected as a prototype, is the method, given in the description of patent U.S. Pat. No. 5,575,818, A 61F2/06, 1995. According to this method a netted hollow volumetric body is formed from the plurality of wire sections, helically wound onto a cylindrical mandrel with opposite entry directions. The elasticity of the stent at its end sections can differ from the elasticity of the central section of the stent due to changing a pitch of wire winding and changing the diameter of the stent. In order to change the diameter of end parts of the stent, it is placed onto a tubular mandrel, while the end sections of the stent are formed by applying deforming efforts to them in a longitudinal direction, then the stent is pre-deformed by heat treatment for attributing a specified form and dimensions to it, corresponding to its operating status and after that the mandrel is removed. This method requires a lesser labour input compared to other analogous methods, but it also does not allow to manufacture stems, the elastic properties of which could be changed at any se=don of the stent, preserving geometrical dimensions of stent cells; it does not provide a sufficient stent rigidity at its end sections and improved transformability, and also does not eliminate the danger of causing traumas to a prostheticated organ when its has a curvilinear form and a small internal diameter. Besides, the technology of the stent manufacturing remains rather complicated.

### The Essence of the Invention

A technical task solved by the proposed variants of the invention, forming a single inventive idea, is the creation of a stent which provides an effective restoration and support of a lumen of hollow organs, including curvilinear, has a good biological compatibility with tissues of a body, minor traumatism, universal physical-and-mechanical and geometric characteristics, big range of typical sizes, providing for a possibility of using small diameter catheters, as well as not requiring a complicated stent delivery system and expensive manufacturing technology.

The above tasks are met by the fact that the cells, opposite to the longitudinal plane of the stent, are made with a displacement with respect to each other, providing for a mismatch of their apexes projections to the above plane, and a stent thread has a different elasticity at its separate sections while preserving the same thickness of the thread along the whole length of the stent, which is new in the known stent, made as a netted hollow volumetric body, formed by interweaving of at least two groups of turns, placed along helical spirals with the opposite entry directions, made of a single length of the thread, the material of which has elasticity and shape memory effect.

In the second variant the basic technical tasks are solved in a principally the same ways the difference lying in the fact that not an elasticity of the thread is changed, but a geometry of the stent by making it with different axial curvature at it separate sections while preserving minimum deviations of geometrical dimensions of cells in curved and rectilinear sections of the stent along all its length, not exceeding 20% with the maximum possible curvature of the stent.

In the third variant the main technical tasks are also solved in a principally the same way, and the difference lies in the fact that it unites the first and the second variants of the stent, that is the stent is made with separate sections having different axial curvature, preserving minimum deviations of geometrical cell dimensions at curvilinear and rectilinear sections of the stent along all its length, not exceeding 20% at the maximum permissible curvature of the stent, and the thread of the stent has different elasticity at its separate sections, preserving the same thickness along the whole length of the stent.

In addition to that, in all three variants the stent is manufactured of nitinol TiNi, containing in weight %: 55.5-56 Ni, the rest being Ti.

In the known method of stent manufacturing, comprising the formation of a netted hollow volumetric body out of a metal thread by way of interweaving its turns, wound onto a mandrel made in the form of a body of revolution with a rectilinear longitudinal aids, pre-deformation of the stent on the mandrel for attributing a specified form and size to it, corresponding to its operating condition, which is carried out by heat treatment, and subsequent removal of the mandrel, new is the fact that the body of the stent is formed out of a single length of a nitinol thread, and the pre-deformation of the stent is carried out by its quenching from the temperature of 630-660° C. into water, which gives a maximum elasticity to this stent, and after the mandrel is removed te elasticity of separate sections of the stents is reduced by secondary heat treatment at the temperature of 330-550° C. during from 1 to 30 minutes, with the temperature and the time of heat treatment being selected in the above ranges based on the condition of their proportionality to the content of Ni in an alloy and inverse proportionality to the value of elasticity specified to separate sections of the stent.

In the second variant of the method the main technical tasks are solved in a principally the same way, the difference being that in it the body of the stent is formed from a single length of a nitinol thread, and pre-deformation of the stent is implemented twice, first by primary heat treatment of the stent on a mandrel with a rectilinear longitudinal axis at the temperature of 330-390° C. during 520 minutes, and upon the end of heat treatment and removal of the above mandrel, the stent is put onto a mandrel with a curvilinear longitudinal axis, the form of which corresponds to the form of a vessel being prostheticated, after which a repeated pre-deformation of the stent by its quenching from the temperature of 630-660° C. into water, which gives a maximum elasticity to this type of the stent, then the mandrel is removed while the temperature and time of heat treatment in the above ranges are selected upon the condition of their proportionality to the content of Ni in an alloy and inverse proportionality to the value of maxim elasticity attributed to this stent.

In the third variant of the method the main technical tasks are also solved in a principally the same way, the difference being that the body of the stent is formed out of a single length of a nitinol thread, and pre-deformation of the stent is carried out twice, first by way of primary heat treatment of the stent on the mandrel with a rectilinear longitudinal axis at the temperature of 330-390° C. during 5-20 minutes, and upon the end of the heat treatment and removal of the above mandrel the stent is put onto the mandrel with a curvilinear longitudinal axis, the form of which corresponds to a vessel being prostheticated, after which a repeated pre-deformation of the stent by way of its secondary heat treatment at the temperature of 380-450° C. during 1-30 minutes takes place and the mandrel is removed; by that, the temperature and the time of heat treatment within the above ranges are selected based upon the condition of their proportionality to the content of Ni in an alloy and inverse proportionality to the elasticity value set to the stent.

In the fourth variant of the method the ma technical tasks are also solved in a principally the same way, the difference being that it unites the first and the second variant of the method, that is the method is characterised by the fa that the body of the stent is formed from a single length of a nitinol tread, and pre-deformation of the stent tes place twice, first by way of a primary heat treatment of the stent on a mandrel with a rectilinear longitudinal axis at the temperature of 330-390° C. during 5-20 minutes, and upon the end of heat treatment and removal of the above mandrel the stent is put onto a mandrel with a curvilinear longitudinal axis, the form of which corresponds to the form of a vessel being prostheticated, after which a secondary pro-deformation of the stent by way of its quenching from the temperature of 630-66° C. into water, which attributes a maximum elasticity to the stent of this type. After removal of the mandrel the elasticity of separate sections of the stent is reduced by their additional heat treatment at the temperature of 330-550° C. during from 1 to 30 minutes, with the temperature and time of heat treatment in the above ranges selected upon the condition of their proportionality to the content of Ni in an alloy and inverse proportionality to the elasticity value specified to separate sections of the stent.

Besides, in all four variants of the method the formation of a body of the stent is carried out with a displacement of cells which are opposite with respect to the longitudinal plane of the stent, providing a mismatch of projections of their apexes to the above plane.

In addition to that in all four variants of the method the stent is manufactured from nitinol TiNi, containing in weight %: 55.5-56 Ni, the rest being Ti.

The advantages of the claimed stent are achieved at the expense of the proposed design and the method of manufacturing the stent.

The displacement of cells which are opposite with respect to the longitudinal plane of the stent, providing a mismatch of projections of their apexes onto the above plane, allows to raise the transformability of the stent and provides for a more tight positioning of the stent in a catheter of a delivery device of the stent, which in its turn allows to decrease the diameter of the catheter and, correspondingly, provide for a possibility of transportation and installation of the stent in organs with a minor internal diameter, as well as to reduce a traumatism of endovascular operations.

Attributing a different elasticity to stent thread at its separate sections while preserving the same thickness of the thread along the whole length of the stent allows to change the elasticity of separate sections of the stent, not spoiling a homogeneity of its structure and geometry of cells, which, on the one hand, permits to create an increased pressure as separate sections of a prostheticated organ when it is required, for examples in case of adenoma, and on the other hand, brig to a minimum the action of additional elastic forces arising from the action of bending moment which appears when there are curvatures in the organ being prostheticated. When an elasticity along the whole length of the stent is equal, its geometrical compatibility with bent sections of prostheticated organs will be achieved only at the expense of the elasticity of the stent and bent sections will be subjected to greater loads than rectilinear ones because of the action of additional elastic forces, which can be the reason of post-operational complications.

The presence in the stent of sections with different axial curvature while preserving minimum deviations of geometrical dimensions of cells in curvilinear and rectilinear sections along all length of the stent, not exceeding 20% at the maximum permissible curvature of the stent, and attributing a different elasticity to a thread of the stent at its separate sections while preserving the same thickness of the thread along the whole length of the stent, allows to provide for an effective stent application also in a specially complicated for prosthetics cases, i.e. when a prostheticated organ or an organ use during the stent delivery to a place of installation has a big curvature or several bents, including in the cases when a small diameter catheter is needed for the delivery and installation of the above stent. By that, all the above advantages from the use of the proposed design features of the stent are reached, as well as the task of providing necessary rigidity to a stent while using a small diameter wire for its manufacturing is solved.

In addition to that the use of nitinol TiNi, containing in weight %: 55.5-56 Ni, the rest being Ti, for the manufacturing of the stent, allows to provide a good biological compatibility with body tissues, as well as simplifies tee delivery system and removal of the stent, The forming of a body of the stent out of a single length of a nitinol tread allows to reduce a traumatism of an operation during an installation of the stent and the probability of post-operational complications, as well as to provide a necessary elasticity of a face sections of the stent.

The use of the proposed heat treatment modes and curvilinear mandrels in manufacturing of the stent permits to provide for the changing of its elastic properties and geometry at the expense of changing elastic properties of the material of the thread while preserving the same thickness and minimum deviations of geometrical dimensions of cells in curvilinear and rectilinear sections of the stent along its whole length.

Pre-deformation of the stent on a mandrel with a rectilinear longitudinal axis by way of its quenching from the temperature 630-660° C. into water provides for giving a specified form and dimensions to the stent, as well as maximum elasticity inherent to its design, material, form and dimensions.

The use of a secondary heat treatment of the stent, conducted after the removal of a mandrel at the temperature of 330-550° C. during from 1 to 30 minutes, allows to reduce the elasticity of separate sections of the stent up to the required value at the expense of changing elastic properties of the material of a thread at these sections.

The selection of temperature and the time of heat treatment in the above ranges upon the condition of their proportionality to the content of Ni in an alloy and inverse proportionality to the value of elasticity specified to separate sections of the stent, allows to achieve a necessary elasticity of the thread in different sections of the stent.

Pre-deformation of the stent on a mandrel with a rectilinear longitudinal axis, made at the temperature of 330-390° C. during 5-20 minutes, allows to attribute specified dimensions to the sent.

Repeated pre-deformation of the stent on a mandrel with a curvilinear longitudinal axis the form of which corresponds to the form of a prostheticated vessel, conducted by quenching the stent from the temperature of 630-660° C. into water, permits to attribute the specified curvilinear form to the stent, while providing a maximum elasticity for this stent.

Repeated pre-deformation of the stent on a mandrel with a curvilinear longitudinal axis, the form of which corresponds to the form of a prostheticated vessel, conducted by quenching the stent from the temperature of 380-450° C. during from 1 to 30 minutes, permits to attribute the specified curvilinear form to the stent, while providing for a elasticity required for this stent, not reaching a possible maximum value, which is sometimes necessary for manufacturing a so-called “soft” stent.

Additional heat treatment of the stent conducted after the repeated pre-deformation of the stent on a mandrel with a curvilinear longitudinal axis by way of quenching the stent from the temperature of 630-660° C. into water and removal of the above mandrel, and carried out at the temperature of 330-550° C. during from 1 to 30 minutes, provides for the reduction of elasticity of separate sections of the stent from a maximum possible value to the required one by changing the elasticity properties of the material of the thread at these sections while the stent has a curvilinear form.

The use of nitinol TiNi, containing in weight %: 55.5-56 Ni, the rest being Ti, for the manufacturing of the stent allows to solve the tasks put by way of using the proposed operations and modes of heat treatment.

### BRIEF DESCRIPTION OF THE FIGURES

The essence of the proposed technical solutions is illustrated by the following drawings:

FIG. 1—a general view of the stent with a rectilinear longitudinal axis;

FIG. 2—a general view of the stent with a curvilinear longitudinal axis;

FIG. 3—a general view of projections of cells which are opposite relative to the longitudinal plane of the stent onto the above plane,

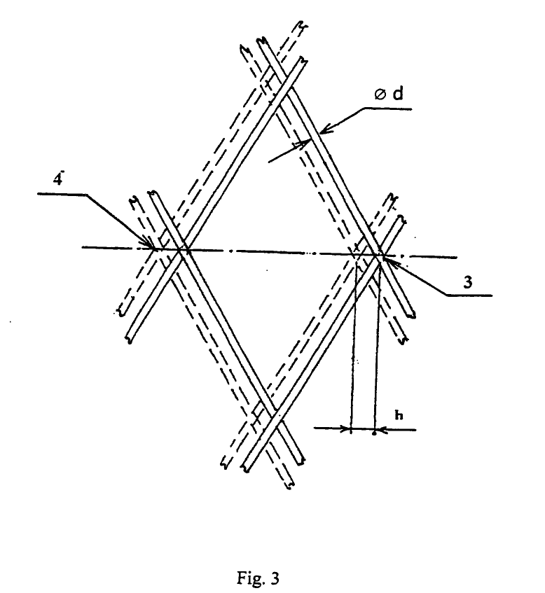

FIG. 4—a general view of the stent and mandrel with curvilinear longitudinal axes in assembly;

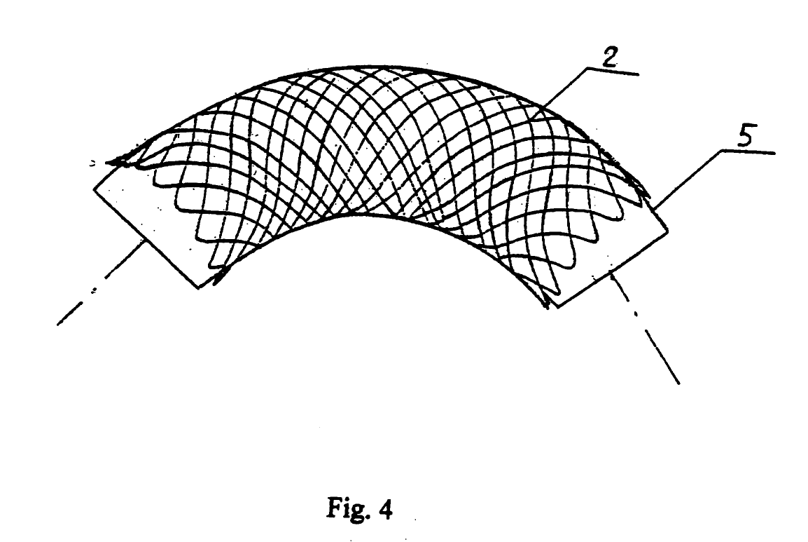

### PREFERRED EMBODIMENTS OF THE INVENTION

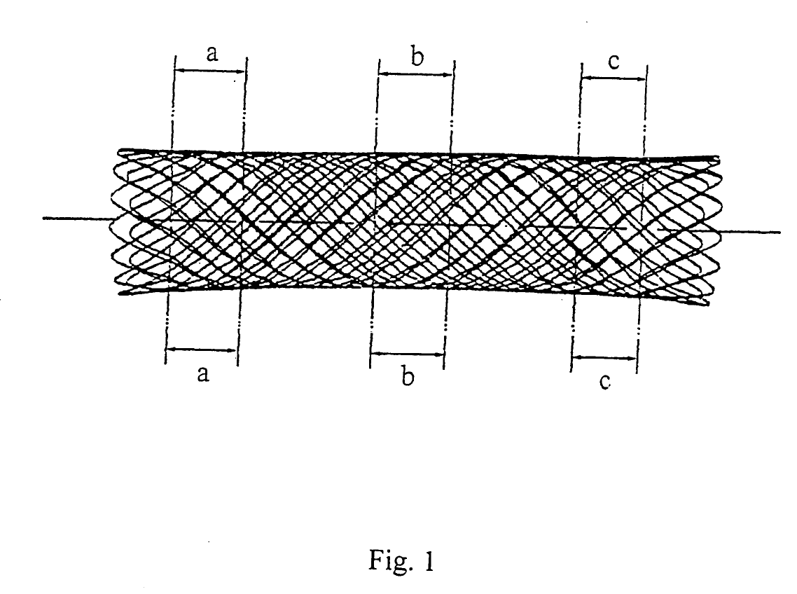

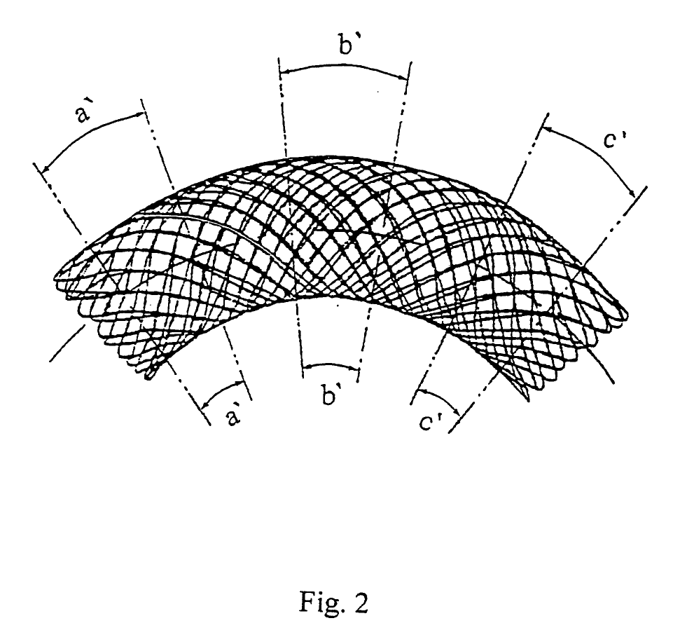

The proposed stent is made as a netted hollow volumetric body 1,2, formed by interweaving of at least two groups of turns, placed along helical spirals with opposite entry directions, made of a single length of a thread, the material of which has elasticity and shape memory effect. Such alloy, in order to be used in a human body, in addition to satisfying an obligatory condition of compatibility with body tissues, should have a temperature for the recovery of the specified form within the ranges from −10 to +40° C. and have a super elasticity effect, appearing at the temperature of above 36° C. It is recommended to use the so-called “medical nitinol”, i.e. TiNi alloy containing in weight %: 55.5-56 NI, the rest being Ti, as such material. Depending on medical requirements, the stent can be made both with a rectilinear longitudinal axis, and with a curvilinear one. Cells 3, 4, which are opposite with respect to the longitudinal plane of the stent, are made with a displacement in respect to each other, providing for a mismatch of projections of their apexes to the above plane. This is related to the peculiarities of this design of the stent in which interweaving of threads, forming apexes of stent cells, contact in overlap. In this case during a radial compression of the stent, happening during its installation into a delivery device, if the above displacement of opposite cells is absent, the total thickness of the stent in the place of their apexes contact will constitute not less than four diameters of the thread. When opposite cells are displaced in one direction along some pair of parallel sides of cells, the total thickness of the stent in the place of cells contact will constitute not less than three diameters of the thread. And only in case of a shift of opposite cells in several directions (if the displacement of cells occurs in the directions parallel to their sides), or in combination of cells displacement with their turning or in case of cells displacement in a diagonal direction, the lower limit of the total thickness of the stent in the place of cells contact can be brought to two diameters of the thread. It is obvious from here that it is possible to achieve a minimisation of the total thickness of the stent in the place of possible contact of opposite cells in different ways. It is only important to provide for a mismatch of apexes projections, which are opposite with respect to the longitudinal plane of the stent, onto the above plane. When the stent is manufactured according to the claimed design, its cells 3,4, have a rhombic form, oriented by one of diagonals along the axis of the sent. For attributing a maximum radial rigidity to this construction it is expedient that smaller diagonals of cells should be oriented along a stent axis, and mismatch of projections of apexes, which are opposite with respect to the longitudinal plane of the stent, should be provided by an opposite cells displacement for distance h along the stent axis. Naturally, the above should not be understood in the sense that under these conditions a mini thickness of the stent within the limits of two tread diameters is obligatorily provided in any place of the stent, as it is not possible to completely exclude the cases, when an apex of some cell of the stent does not get into contact with the side of an opposite cell during radial compression. However, when the stent is made with a displacement of cells which are opposite with respect to the longitudinal plane of the stent relative to each other, providing for a mismatch of their projections to the above plane, it is possible to bring such probability to a minimum, achieving the improvement of the stent transformability and the increase of its density while laying into a delivery device. The stent is manufactured in several variants, the selection of which is determined by conditions of a specific operation. In the first recommended variant different elasticity is given to threads of the stent at its different sections (for example, in sections a,b,c, FIG. 1), preserving the same thickness of the stent along its whole length. In the second variant the stent is characterised by its having separate sections with different axial curvature, while preserving minimum deviations of geometrical dimensions of cells in curvilinear and rectilinear sections of the stent along its whole led, not exceeding 20% with the maximally permissible curvature of the stent. In the third variant of the stent the presence of different axial curvature in separate sections of the stent is combined with attributing a different elasticity to a thread of the stent at its separate sections (for example, in sections a′, b′, c′, FIG. 2) while preserving the same thickness of the thread along the whole length of the stent. Depending on peculiarities of the design of the stent several variants of its manufacture are proposed.

In the first variant of the method, meant for the manufacturing of a rectilinear stent with different elasticity of a thread in its different sections, a nitinol thread is wound onto a mandrel, made in the form of a body of revolution with a rectilinear longitudinal axis, and equipped with guides se a required pitch to a spiral winding. In the process of braiding at least two groups of turns, placed along helical spirals with the opposite entry directions, are interwoven, forming by this a netted hollow volumetric body of the stent with a specified form and dimensions, corresponding to its operational status. The displacement of stent cells which are opposite with respect to the longitudinal plane of the stent, is implemented by way of a shift of the guides along the axis of the stent, using places of a thread interweaving nodes location on a mandrel and, correspondingly, the location of stent cells apexes, and providing a specified pitch for the spiral winding. By that, in order to provide for a mismatch of apexes projections, which are opposite with respect to the longitudinal axis of the stent, onto the above plane, it is enough to shift the guides in the axial direction in such a way that their shift h in a diametrically opposite points of the mandrel would constitute 1.2-1.5 of the diameter of the thread. Moreover, as it was experimentally established, the above shift of the guides causes not only a displacement of opposite stent cells in the axial direction, but also their turning, which is the reason of a slight reduction of the radial rigidity of the stent which constitutes not more than 5% from the radial rigidity, of the stent before cells turning at the above shift, which is easily compensated by the increase of the elasticity of the thread as a result of subsequent heat treatment. After the end of the formation of the body of the stent, in order to preserve and store its forms and dimensions, the stent is subjected to pre-deformation, accomplished by stent quenching from the temperature of 630-660° into water, which attributes a maximum elasticity to this stent, determined by the elasticity of the thread (E≈83400 MPa E—Young's modulus of the thread material, MPa—mesa Pascal). Then the stent is released from the mandrel and the elasticity of separate sections of the stent is reduced by their secondary heat treatment at the temperature of 330-550° C. ding from 1 to 30 minutes. The heat treatment can be carried out using conventional circular type heaters with a specified diameter and width, which allows to reduce the elasticity of the fad in separate sections of the stent up to a specified value (up to E≈54000-58900 MPa). Such stent is recommended to eliminate a stenosis caused by e n pressure upon separate sections of a prostheticated organ (for example, in case of adenoma) and/or in case of relatively small curvatures of a prostheticated organ.

In the second variant of the method, meant for manufacturing a curvilinear stent having a maximum for this stent elasticity, the formation of the body of the step is accomplished in several stages. At the first stage, similarly to the first variant of the method, a netted hollow volumetric body of a stent with specified dimensions is formed using a mandrel, made in the form of a body of revolution with a rectilinear longitudinal axis. After finishing the first stage of the formation of the body of the stent, the stent is subjected to pre-deformation for preserving and storing the form and dimensions obtained, which is accomplished by way of a heat treatment of the stent on the mandrel with a rectilinear longitudinal axis at the temperature of 330-390° C. during 5-20 minutes. At that the stent is released from the above mandrel and is put on mandrel 5 with a curvilinear longitudinal axis, the form of which corresponds to the form of a prostheticated vessel, after which a repeated pre-deformation of the stent takes place by way of its quenching from the temperature of 630-660° C. into water which brings about a maximum elasticity for this type of the stent, and then the mandrel is removed. The use of two mandrels in this variant for the manufacturing of the stent is determined by great technological difficulties which arise in the attempt to from the body of the stent by winding and interweaving directly on a curvilinear mandrel. The stent manufactured according to this method is recommended to be used when there are considerable curvatures in a prostheticated organ.

In the third variant of the method, meant for the manufacturing of a curvilinear stent having a specified elasticity, not reaching a possible maximum value, the formation of the body of the stent is also happening in several stages. At the first stage, similarly to the first and the second variant of the method, a netted hollow volumetric body of the stent of specified dimensions is formed using a mandrel made as a body of revolution with a rectilinear longitudinal axis. After the end of the first stage of the formation of the body of the stent it is subjected to pre-deformation for preserving and storing an obtained form and dimensions, which is carried out by way of heat treatment of the stent on a mandrel with a rectilinear longitudinal axis at the temperature of 330-390° C. during 5-20 minutes. After that the stent is released from the above mandrel and put on a mandrel with a curvilinear longitudinal axis, the form of which corresponds to te form of a vessel being prostheticated after which a repeated pre-deformation of the stent takes place by way of its secondary heat treatment at the temperature of 380-450° C. during from 1 to 30 minutes and then the mandrel is removed. It is recommended to use a curvilinear stent, which can be called a “soft stent”, manufactured according to this method, in cases when a maximum rigidity is not required from the stent, for example, for eliminating the consequences of a balloon dilatation of arteries with expressed curvature, during which a rupture and detachment of intima often occurs. Other example can be the cases of conducting an operation in such area of a body where there are difficulties in controlling the course of operation with a help of x-ray radiation, for example in an abdominal cavity, especially in fat patients. In this case, if tere are no other restrictions, it might be expedient to use a stent with a somewhat bigger cross-section, which allows to provide for a better visibility of the stent in an x-ray radiation, reducing an extra elasticity of the stent when required.

In the fourth variant of the method, meant for manufacturing of a curvilinear stent with a different elasticity at its different sections, the formation of the body of the stent is also accomplished in several stages. At the first stage, similarly to the preceding variant of the method a netted hollow volumetric body of the stent of specified dimensions are formed using a mandrel made as a body of revolution with a rectilinear longitudinal axis. After finishing the first stage of the formation of the body of the stent, it is subjected to pre-deformation for preserving and storing a form and dimensions obtained, which is accomplished by way of heat treatment of the stent on a mandrel with a longitudinal axis at the temperature of 330-390° C. during 5-20 minutes. After that the stent is released from the above mandrel and is put onto a mandrel with a curvilinear longitudinal axis, the form of which corresponds to the form of a prostheticated vessel, after which a repeated pre-deformation of the stent by quenching it from the temperature of 630-660° C. takes place, which gives a maximum elasticity for this stent, determined by the elasticity of the thread (E≈83400 MPa). Then the stent is released from the mandrel and the elasticity of separate sections of the stent is decreased, subjecting them to a secondary heat treatment at the temperature of 330-550° C. during from 1 to 30 minutes. The heat treatment, similarly to the first method, can be conducted with the use of conventional ring type heaters of specified diameter and width, which allows to decrease the elasticity of the thread at separate sections of the stent up to a specified value (up to E≈54000-58900 MPa). Such stent is recommended for the use in a specially complicated configuration of a prostheticated organ.

In all the methods the body of the stent is formed from a single length of a metallic wire made of “medical nitinol”, that is the alloy TiNi, containing in weight %: 55.5-56 Ni, the rest being Ti. The temperature and the time of heat treatment in the above ranges are selected during the manufacturing of the stent based on the condition of their proportionality to the content of Ni in an alloy and inverse proportionality to the value of elasticity specified to separate sections of the stent. The above relationships were set experimentally for the “medical nitinol”, i.e. for the nitinol allow with a given content of components. Thus, for example, in the fourth variant of the method while attributing a elasticity at which E≈58700 MPa to the material of the at separate sections of the stent, depending on the content of nickel in the alloy, the temperature and the time of heat processing were selected in the following ranges:

TABLE 1

| Content of | Temper- | Temper- | | |
|---|---|---|---|---|
| nickel in | ature of | ature of | Time of heat treatment, min | |
| an alloy in | annealing | quenching | Primary | Secondary |
| weight % | ° C. | ° C. | heat treatment | heat treatment |
| 55.5 | 330-390 | 630-640 | 5-10 | 1-10 |
| 55.7 | 390-450 | 640-650 | 10-15 | 10-15 |
| 56.0 | 450-550 | 650-660 | 15 20 | 15-30 |

The proposed technology of stent manufacturing is not limited to manufacturing stents of the proposed design, and can be used for manufacturing nitinol stents of other designs. However, the greatest efficiency of the claimed technology is revealed in mauling stents of the proposed design namely.

The operation of the device is carried out in the following way. A prostheticated organ is catheterised in the place of the installation of a stent and stenosis is removed if required. The stent, manufactured from a medical nitinol, is cooled up to the temperature, equal or lesser than the temperature of the beginning of a direct martensite transformation in the nitinol (the temperature of cooling for the recommended composition of the thread material is selected in the range from −5 to +15° C.), for example, by processing with chloroethyl, then drawn in an axial direction for attributing a minimum diameter to it, and placed into the delivery device. When the stent is drawn, its threads are sliding with respect to each other, which in combination with a specified displacement of opposite cells with respect to each other allows to minimise the diameter of catheters used in operation without introducing irreversible damages into the structure of the stent and changing the thickness of the thread. Known devices can be used as a delivery device for the stent implantation, such, for example, as described in patent RU 2149037, A61M25/0, 1997. Then the delivery device with the stent are delivered with a help of a catheter to the place of prosthetics under a permanent x-ray control, after which the stent is released and the delivery device is removed. Under the influence of the temperature of a body the stent starts to be heated, the temperature of the stent thread becomes higher than that of the point of a nitinol martensite transformation, and the stent restores an initially set form for several seconds in the process of the release. After that the stent, interacting with walls of a prostheticated organ, for example, an artery, maintains a lumen and geometry unchanged due to its elasticity dig necessary time.

### Industrial Applicability

The proposed stent can find a wide application in the field of medicine, using endoprosthetics for restoring a lumen in narrowed sections of vessels and other hollow organs, as it provides an effective restoration and support of the lumen of hollow organs, including curvilinear ones, and has a good biological compatibility with tissues of an organism, low traumatism, universal physical-and-mechanical and geometrical characteristics, a big range of typical sizes, providing for the use of catheters of small diameter, as well as does not require a complicated stent delivery system.

The design of the stent allows it to be sufficiently well adjusted to the geometry of hollow organs, including those having a big curvature, to bring down to a minimum undesirable action of elastic forces, arising during bending of the stent.

The proposed method of stent manufacturing allows to provide a serial production of the stents of the claimed design, achieving claimed characteristics. By that, it does not require an expensive equipment and complicated manufacturing technology, as well as it also allows to manufacture stents adapted to individual peculiarities of a prostheticated organ of a concrete patient.

The above peculiarities of the proposed stent and the method of its manufacturing contribute to the creation of necessary conditions for a mass application of endoprosthetics in treatment of hollow organs diseases.

The results of the use of the claimed device can be illustrated by the following practical example.

### EXAMPLE

Patient K., 63 years old, was for a long time suffering from arteriosclerosis of the main arteries of a pelvis and low extremities. During the last month up to a hospitalisation the symptoms of an intermittent claudication from the side of a right low extremity—pains in musculus gastrocnemius of the right crus was appearing after walking for 50-70 meters.

During arteriography of the pelvis a stenosis of the right common ileal artery was revealed. A balloon dilatation of the stenosis of this artery was performed. However after a balloon catheter was removed, a considerable residual stenosis of the dilated artery was observed dung a control arteriography of the pelvis. In this connection a month after a nitinol stent of the claimed design with the size of 10×60 mm and the thickness of the thread of 0.2 mm was implanted to the patient into hew area of the stenosis. During the arteriography of the pelvis, made after the operation, a complete liquidation of the stenosis was observed. The symptoms of intermittent claudication from the side of the low extremity disappeared already on the next day. After a short course of a desagregational therapy the patient was released for an out patient treatment. In a control arteriography of the pelvis, made in 8 months, a complete permeability of the right crus was pointed out. The nitinol stent was covered by a thin layer of neointima from the inside.

1. A stent made in the form of a netted hollow volumetric body, formed by interweaving of at least two groups of windings, placed along helical spirals with opposite entry directions, made of a single length of a thread, the material of which possesses elasticity and shape memory effect, wherein cells, opposite in relation to the longitudinal plane of the stent, are displaced with respect to each other, providing for a mismatch of projections of their apexes to the above plane, and the stent thread has a different elasticity in its separate sections, preserving the same thickness of the thread along all length of the stent.

2. The stent made in the form of a netted hollow volumetric body, formed by interweaving of at least two groups of windings, placed along helical spirals with opposite entry directions, made of a single length of a thread, the material of which possesses elasticity and shape memory effect, wherein the cells, opposite in relation to the longitudinal plane of the stent, are displaced with respect to each other, providing for a mismatch of projections of their apexes to the above plane, the stent being made with its separate sections having a different axial curvature preserving minimum deviations of cells geometrical dimensions along curved and rectilinear sections of the stent, not exceeding 20% at a maximum permissible curvature of the stent, along the whole length of the stent.

3. The stent made in the form of a netted hollow volumetric body, formed by interweaving of at least two groups of windings, placed along helical spirals with opposite entry directions, made of a single length of a thread, the material of which possesses elasticity and shape memory effect, wherein cells, opposite in relation to the longitudinal plane of the stent, are displaced with respect to each other, providing for a mismatch of the projection of their apexes to the above plane, and the stent thread has a different elasticity at its separate sections, while preserving the same thickness along the whole length of the stent; the stent being made with its separate sections having a different curvature preserving minimum deviations of cells geometrical dimensions along curved and rectilinear sections of the stent, not exceeding 20% at a maximum permissible curvature of the stent, along the whole length of the stent.

4. The stent of claim 1, or claim 2, or claim 3, wherein it is manufactured from nitinol TiNi, containing in mass %: 55.5-56 Ni, the rest Ti.

5. A method of manufacturing a stent, including the formation of a netted hollow volumetric body from a metallic thread by interweaving its windings, woven on a mandrel made as a body of revolution with a rectilinear longitudinal axis, pre-deformation of the stent on the mandrel for giving to it a specified form and dimensions corresponding to its operational status by means of heat treatment and subsequent removal of the mandrel, wherein the body of the stent is formed from a single length of a nitinol thread, and the pre-deformation of the stent is performed by quenching it from the temperature of 630-660° C. into water, attributing a maximum elasticity for this stent; after removal of the mandrel the elasticity of separate sections of the stent is reduced by their secondary heat treatment at the tempera of 330-550° C. during from 1 to 30 minutes, with the temperature and the of heat treatment in the above ranges being selected upon the condition of their proportionality to the content of Ni in the alloy and inverse proportionality to the value of elasticity specified to different sections of the stent.

6. The method of manufacturing a stent, including the formation of a netted hollow volumetric body from a metallic thread by interweaving its windings, woven on a mandrel made as a body of revolution with a rectilinear longitudinal axis, pre-deformation of the stent on the mandrel for giving to it specified form and dimensions corresponding to its operational status by means of heat treatment and subsequent removal of the mandrel, wherein the body of the stent is formed from a single length of a nitinol thread, and the pre-deformation of the stet is performed twice, first by an initial heat treatment of the stent on the mandrel with a rectilinear longitudinal axis at the temperature of 330-390° C. during 5-20 minutes, and after the end of heat treatment and removal of the above mandrel the stent is put onto a mandrel with a curvilinear longitudinal axis, the form of which corresponds to the form of prostheticated vessel, after which a repeated pre-deformation of the stent is performed by quenching it from the temperature of 630-660° C. into water, which attributes a maximum elasticity to this stent; and the mandrel is removed, with the temperature and time of heat treatment in the above ranges selected upon the condition of their proportionality to the content of Ni in an alloy and inverse proportionality to the value of maximum elasticity, attributed to this stent.

7. The method of manufacturing a stent, including the formation of a netted hollow volumetric body from a metallic thread by interweaving its windings, woven on a mandrel made as a body of revolution with a rectilinear longitudinal axis, pre-deformation of the stent on the mandrel for giving to it specified form and dimensions corresponding to its operational status by means of heat treatment and subsequent removal of the mandrel, wherein the body of the stent is formed from a single length of a nitinol thread, and the pre-deformation of the stent is performed twice, first by an initial heat treatment of the stent on the mandrel with a rectilinear longitudinal axis made at the temperature of 330-390° C. during 5-20 minutes, and after the end of heat treatment and removal of the above mandrel the stent is put onto a mandrel with a curvilinear longitudinal axis, the form of which corresponds to the form of prostheticated vessel, after which a repeated pre-deformation of the stent is performed by way of its secondary heat treatment at the temperature of 380-450° C. during from 1 to 30 minutes and removing the mandrel, while the temperature and time of heat treatment in the above ranges are selected upon the condition of their proportionality to the content of Ni in the alloy and inverse proportionality to the value of elasticity, attributed to this stent.

8. The method of manufacturing a stent, including the formation of a netted hollow volumetric body from a metallic tread by interweaving its windings, woven on a mandrel made as a body of revolution with a rectlinear longitudinal axis, pre-deformation of the stent on the mandrel for giving to it specified form and dimensions corresponding to its operational status by means of heat treatment and subsequent removal of the mandrel, wherein the body of the stent is formed from a single length of a nitinol thread, and the pre-deformation of the stent is performed twice, first by an initial heat treatment of the stent on the mandrel with a rectilinear longitudinal axis made at the temperature of 330-390° C. during 5-20 minutes, and after the end of beat treatment and removal of the above mandrel the stent is put onto a mandrel with a curvilinear longitudinal axis, the form of which corresponds to the form of prostheticated vessel, after which a repeated pre-deformation of the stent is performed by way of its quenching into water from the temperate of 630-660°, attributing a maximum elasticity for this stent, and after the removal of the mandrel the elasticity of separate sections of the stent is reduced by additional heat treatment at the temperature of 330-550° C., during from 1 to 30 minutes, while the temperature and time of heat treatment in the above ranges are selected upon the condition of their proportionality to the content of Ni in the alloy and inverse proportionality to the value of elasticity, attributed to separate sections of the stent.

9. The method of claim 5, or claim 6, or claim 7, or claim 8, wherein the formation of the body of the stent is made with a displacement of opposite with respect to the longitudinal plane of the stent cells, providing for a mismatch of projections of their apexes upon the above plane.

10. The stent of claim 5, or claim 6, or claim 7, or claim 8, wherein it is manufactured from nitinol TiNi, containing in mass %: 55.5-56 Ni, the rest Ti.
